(19) United States
(12) Patent Application Publication
(10) Pub. No.: US 2004/0022673 A1
(43) Pub. Date: Feb. 5, 2004

(54) Sterilisation process and apparatus therefor

(76) Inventor: Jacques Protic, Anglesey (GB)

Correspondence Address: DENNISON, SCHULTZ & DOUGHERTY, 1745 JEFFERSON DAVIS HIGHWAY, ARLINGTON, VA, 22202, US

(21) Appl. No.: 10/332,992

(22) PCT Filed: Jul. 26, 2001
(86) PCT No.: PCT/GB01/03378

(51) Int. Cl. A61L 2/14

(52) U.S. Cl. 422/28; 422/30; 422/22; 422/292; 422/186.05; 422/186.08

(57) ABSTRACT

The invention provides a sterilization process which is not carried out in a vacuum, including the steps of introducing an oxidizing agent in a gaseous state into a sterilization chamber or area to be sterilized, and introducing gas plasma into the sterilization chamber or area to be sterilized. An apparatus for carrying out the process is disclosed.

The present invention relates to a sterilisation process. In particular, the present invention relates to a sterilisation process that does not need to be carried out in a vacuum. More particularly, the present invention relates to a gas plasma sterilisation process carried out at atmospheric, or a slightly positive, pressure. Additionally, the present invention relates to an apparatus therefor.

As will be appreciated, it is imperative that materials and devices used for the practice of medicine are aseptic.

As a result thereof, much research has gone into the development of processes suitable for effecting sterilisation of such materials and devices.

Generally, such processes are carried out by the manufacturer of such materials and devices, or by users thereof, for example, by medical establishments, such as hospitals.

One sterilisation process currently being utilised involves exposing such materials and devices to ethylene oxide gas for at least one hour and then leaving same to aerate for a minimum period of twelve hours. As is well documented, one of the major problems associated with the use of ethylene oxide gas is that it is highly toxic and dangerous to humans, that is, it is a known carcinogen and mutagen. Additionally, and due to the aeration time required, it is time consuming.

Another low temperature sterilisation process involves treating, by irradiation, the materials and devices with Gamma radiation. Although such process overcomes the disadvantages associated with the use of ethylene oxide, it will be appreciated that the apparatus therefor is extremely expensive and more importantly, it cannot be used to sterilise any electro medical devices or items, since any item including a memory chip is simply wiped clean by the action of Gamma radiation thereon.

Another sterilisation process involves the use of steam autoclaving. As will be appreciated by those skilled in the art, one of the problems associated with the use of such a process is that it requires high temperatures and therefore, is not suitable for sterilising materials or devices made out of matter that is affected by either moisture or high temperature, i.e. its application is fairly limited and depends on the inherent nature of the matter out of which the material or device to be sterilised is made.

With a view to overcoming the problems associated with the sterilisation processes outlined above, a number of low temperature sterilisation processes, involving the use of gas plasma, have been developed.

One of the first sterilisation systems involving the use of gas plasma that received approval from the Food and Drug Administration (FDA) was the STERAD sterilisation system manufactured by Advanced Sterilisation Products, a division of Johnson and Johnson. Such system operates as follows:

Items to be sterilised (the “load”) are placed into the sterilisation chamber of the STERAD steriliser and air is evacuated therefrom to produce a vacuum. When a sufficiently low pressure is achieved within the chamber, a low temperature gas plasma is generated to aid with the removal of any residual moisture from the items being sterilised. This is known as the pre-plasma stage.

At the end of the pre-plasma stage, the system is vented to atmospheric pressure by the introduction of filtered air. This process step constitutes the end of the pre-treatment drying phase, and the sterilisation process then begins.

To start sterilisation, the pressure within the chamber is reduced and an aqueous solution of hydrogen peroxide is injected into and vaporized within the chamber. The resulting hydrogen peroxide vapour diffuses through the chamber thereby surrounding the items to be sterilised and initiating the inactivation of the microorganisms.

The pressure within the chamber is then increased, and then following a subsequent pressure reduction, a low temperature plasma is generated by applying RF energy to create an electric field that in turn initiates the generation of the plasma. In the plasma, the hydrogen peroxide vapour is broken apart into reactor species known as free radicals. After the activated components react with the organisms, other materials, or each other, they lose their high energy and recombine to form oxygen, water vapour and other non-toxic by-products. This constitutes one half of the total sterilisation process, which is then completed by repeating the above steps, that is, with the exception of repeating the pre-treatment drying phase.

At the completion of the second half cycle, the RF energy is turned off, the vacuum is released, and the chamber is returned to atmospheric pressure by the introduction of HEPA-filtered air.

Although widely used, the STERAD apparatus and process exhibits the following disadvantages:

A. Moisture interferes with the STERAD steriliser's ability to attain vacuum conditions. As a result thereof, the presence of excess moisture will result in the STERAD apparatus aborting the sterilising process being carried out therein;

B. Sterilisation will not occur if any organic material is present, since the sterilising agent will decompose. This results in the necessity of ensuring that the items to be sterilised are thoroughly clean before being placed in the STERAD steriliser. As will be appreciated, this is time consuming;

C. The STERAD steriliser cannot sterilise items having long (greater than 12 inches) or narrow lumens without the assistance of a “diffusion intensifier”. As will be appreciated, such a constraint limits the STERAD steriliser's utility;

D. The items to be sterilised can only be packaged, or wrapped, in polypropylene sterilisation wrap or polypropylene pouches. In this connection, the STERAD system cannot achieve sterilisation of items wrapped in tear-proof paper, which is the standard packaging material used extensively in hospitals. The reason being is that the hydrogen peroxide, which is in droplet form, would be absorbed by the tear proof paper wrapping thereby making the STERAD process ineffective. As a result thereof, it is recommended that users of the STERAD steriliser use a special and considerably more expensive synthetic wrapping made by Du Pont and sold under the trade mark, Tyvek®;

E. As with other sterilisation processes involving the use of hydrogen peroxide, liquids, powders and absorptive materials, such as paper, cannot be sterilised due to their tendency to absorb hydrogen peroxide. As will be appreciated, such absorption hinders sterilisation efficiency and aeration of the sterilant;

F. As a vacuum is required to carry out the STERAD process, the size of the sterilising chamber is limited. As will be appreciated, this makes the STERAD steriliser suitable for relatively small volume process applications only; and

G. It has been documented that hydrogen peroxide is not as penetrating as ethylene oxide.

According to the present invention, there is provided a sterilisation process which is not carried out in a vacuum including the steps of:

introducing at least one oxidising agent into a sterilisation chamber or area to be sterilised; and

introducing gas plasma into the sterilisation chamber or area to be sterilised.

It is believed that the sterilisation process of the present invention at least addresses some of the disadvantages associated with the STERAD process outlined above. In particular, and since the process of the present invention is not carried out in a vacuum, the presence of small traces of moisture does not inhibit sterilisation. Moreover, as the sterilisation process of the present invention does not need to be carried out in a vacuum, it can be utilized for large volume applications, for example, it can be utilised to sterilise laboratory rooms, bio-hazardous areas and similar environments or enclosures. Furthermore, the process of the present application is still effective even when the materials or devices to be sterilised are soiled. In this connection, and during our investigations, we smeared items to be sterilised with quick drying protein and observed that same had no affect on the effectiveness of the process of the present invention. Consequently, it is believed that the process of the present invention is more penetrating and moreover, does not require a pre-sterilising check, which is time consuming, to ensure that the items to be sterilised are thoroughly clean.

The stages of the process of the present invention will now be described separately hereinbelow:

Stage 1

Stage 1 involves the addition of an oxidising agent, preferably in a gaseous or vaporised state, into the sterilisation chamber or area to be sterilised. Such oxidising agent starts the sterilisation process by interacting with the many proteins and nucleic acid groups found in the outer coatings, for example, cell walls and cell membranes, of infectious biological agents, such as bacterium, fungi and viruses etc. likely to be present on the surgical apparatus or instruments (the “load”) being sterilised. That is, such an oxidising agent affects the integrity of such outer coatings thereby weakening the biological agents defences to the sterilising action of the present process.

In a preferred embodiment, the oxidising agent is an aldehyde, preferably, formalin, that is, formaldehyde in gaseous or vaporised state. Further preferably, the formalin used is in concentrations of 10-40% ww, preferably 35% ww, and is added in a ratio of 1-10 ml, preferably 3 ml, per cubic metre of effective sterilisation chamber, or area to be sterilised, volume. One of the main advantages of using formalin is that same can be utilised with both standard paper wrapping and synthetic specialised wrappings like Tyvek®, since formaldehyde is readily evaporated by exposure to elevated temperatures.

In an alternative preferred embodiment, the oxidising agent utilised is peracetic acid (peroxyacetic acid). Preferably, the peracetic acid is used in concentrations of 3-20% ww, preferably 12% ww, and is added in a ratio of 1-10 ml, preferably 3 ml, per cubic metre of effective sterilisation chamber, or area to be sterilised, volume.

In a preferred embodiment, prior to introducing gas plasma into the sterilisation chamber or area to be sterilised, the oxidising agent is re-circulated within the sterilisation chamber or area to be sterilised for a period of 1 to 20 minutes, preferably 5 to 15 minutes, further preferably 10 minutes. It is believed that such re-circulation provides optimum initial exposure of the oxidising agent to the microorganisms to be eradicated.

Stage 2

The second stage of the process of the present invention involves the introduction of a high energy gas plasma into the chamber or area to be sterilised.

In general terms, the plasma can be described as a highly ionised gas in which the number of free electrons is approximately equal to the number of positive ions.

The gas plasma may be derived from ambient air, with or without the presence of other gases, and with or without oxygen concentrators. The gas plasma is a partially ionized gas composed of ions, electrons, and neutral species. Such a state of matter can be produced through the action of either very high temperatures or strong electric or magnetic fields. Preferably, the ionised gas is produced by gaseous electric discharges from dry compressed air at a constant pressure of between about 4-8 bar, preferably 6 bar and continuous airflow of 5-20, preferably 12 litres per minute. Preferably, the pressure inside the sterilisation chamber or area to be sterilized during the gas plasma phase is lower and is controlled to 1-100, preferably 10, pascals above atmospheric.

In an electrical discharge, free electrons gain energy from the imposed electric field and lose this energy through collisions with neutral gas molecules. The energy transfer process leads to the formation of a variety of highly reactive products including meta-stable atoms, free radicals and ions. For instance, in an oxidising plasma some of the active species are the atomic oxygen and '.DELTA.g molecular oxygen which is also called “singlet oxygen”. A singlet molecule is one in which the absorption of energy has shifted a valence electron from its normal bonding orbital to an antibonding orbital of higher energy, and in which the electron spins are paired (oxygen is an unusual diatomic molecule in that the spins of the two valence electrons of lowest energy are not paired in the most stable or ground state). The resultant excited molecule is highly unstable and must release its excess energy through different pathways or re-combinations. Artificially generated singlet oxygen reacts with regions of high electron density in microbial substrates. For an ionized gas produced in an electrical discharge to be properly termed a plasma, it must satisfy the requirement that the concentrations of positive and negative charge carriers are approximately equal.

It has been found quite surprisingly that small amounts of vaporized oxidising agents and free radicals present in the low temperature gas plasma greatly increases the overall biocidal action of the gas plasma, particularly, if the required conditions are generated under a positive pressure regime.

To this end, and by way of explanation, when an aldehyde, like formalin, is exposed to high-energy gas plasma it is believed that H—C═O. radicals form, which will react with oxygen to form a radical intermediate. This in turn will react with formaldehyde to produce performic acid and ultimately, formic acid.

H—C═O.+O₂→H—C═O.OO.+H₂C═O→HC═O.OOH (performic acid)→H—C═O.OH (formic acid)

Due to the presence of atomic or excited oxygen in the gas phase, the formalin can produce very reactive short-life epoxides which can interact with many proteins and nucleic acids groups in the outer layer coats of spores to enhance sporicidal activity. This action is also highly effective against non-sporulated bacteria.

The sporicidal activity of formalin seems directly related to the concentration of free aldehyde radicals. In addition, the oxidation of formaldehyde gas into formic acid (HCOOH) as a vapour increases the sporicidal efficacy of epoxides. In the case of formalin, there is a very favourable set of conditions to attack the spore outer layers with active oxygen and facilitate several types of reactions with proteins, while also increasing the penetration and density of formaldehyde radicals in the critical areas of the spore. As such, a low temperature oxidising gas plasma seeded with formalin will provide numerous reactive intermediates and free radicals to alter the spore outer coats and thus improve the diffusion of bactericidal groups. This mechanism explains why short exposures to a plasma gas in the presence of formaldehyde (formalin) can quickly destroy spores of their germinating capabilities. While excited ions, gas molecules and photons profoundly modify the protective layers of the spores, the active formaldehyde radicals affect the cell structures and initiate many additional lethal reactions which accelerate the killing process.

It is to be understood that within the context of the present invention the introduction of gas plasma into the sterilisation chamber or area to be sterilised includes generating the gas plasma within the sterilisation chamber or area to be sterilised and/or introducing the gas plasma into the sterilisation chamber or area to be sterilised from an outside source. It is also to be understood that the gas plasma can be generated from air and/or oxygen and/or any other oxidising agent in a gaseous or vaporised state.

In a preferred embodiment, and once sterilisation is complete, the process in accordance with the present invention further includes a neutralising or purging stage to neutralise any harmful residues that may remain within the sterilisation chamber or area to be sterilised. To this end, neutralisation may be effected by drawing air out of the sterilisation chamber or area to be sterilised and passing same through a carbon filter. In addition, or instead of, it is preferable that the sterilisation chamber or area to be sterilised is then flushed with air drawn through a ULPA or HEPA filter. In addition, and in the situation where the area to be sterilised is a room, for example, a hospital ward, it is also preferable to add ozone to neutralise any harmful residue remaining within the area to be sterilised, that is, once sterilisation is complete. For example, and when formalin is introduced into the area to be sterilised, ozone will convert any formalin gas residue absorbed by any articles capable of absorbing formalin within the room, such as sheets, into carbon dioxide and water.

In a preferred embodiment, the plasma is produced or generated for 5-180 minutes, preferably 5-90 minutes, further preferably 545 minutes, particularly preferably 20 minutes. It is believed that such duration is sufficient to enable the required interaction between the free radicals produced within the plasma field and the vital cell components, such as cell membranes, enzymes and nucleic acids, such that the life functions of the organisms to be eradicated is disrupted.

Further preferably, during the plasma producing stage, gas within the sterilisation chamber or area to be sterilised is re-circulated to give maximum exposure. In addition, re-circulation has the advantage of attaining total mixing of the gaseous contents within the chamber or area to be sterilised and/or to assist in pushing any active gas through any long lumen devices present within the load. In this connection, it is to be understood that any lumen containing devices within the load, particularly long lumen devices, may actually be connectable to the supply or extract/exhaust ports of the re-circulation system such that the sterilising gases can be drawn therethrough. Furthermore, it is to be understood that during such re-circulation the gas can be passed or re-circulated through the gas plasma generating means, for example, a gas plasma generator.

Further preferably, and during the production of plasma, additional oxygen electrons or free radicals may be introduced into the sterilisation chamber or area to be sterilised. Preferably, this is effected by drawing air and/or oxygen into the sterilisation chamber or area to be sterilised, preferably through the gas plasma generator.

In a preferred embodiment, during the production of plasma, the temperature within the sterilisation chamber or area to be sterilised is maintained from 25° C. to 66° C., preferably at 50° C. or within +/−3° C. thereof. By varying the temperature, it will be appreciated that the internal pressure within the sterilisation chamber or area to be sterilised will correspondingly change thereby enabling better penetration of items that need to be sterilised; even items which are wrapped.

In a preferred embodiment, the temperature within the sterilisation chamber or area to be sterilised prior to the production of plasma is maintained (“the pre-plasma stage”) from 22° C. to 45° C. During our investigations we noted that an increase of temperature prior to the introduction of the gaseous sterilising agent, in particular, formalin, improved the effectiveness of same. More particularly, we observed that the effectiveness of formalin was increased when the sterilisation chamber or area to be sterilised was maintained at a temperature of 45° C., particularly when the load wasn't wrapped.

In a further aspect of the present invention there is provided a sterilisation apparatus when used to carry out the sterilisation process in accordance with the present invention, the sterilisation apparatus being provided with a sterilisation chamber or being connectable to an area to be sterilised and including:

means for providing the sterilisation chamber or area to be sterilised with an oxidising agent in a gaseous state; and

means for introducing or generating plasma within the sterilisation chamber or area to be sterilised.

In a preferred embodiment, the plasma generating means are isolated such that they cannot come into contact with the items to be sterilised. This has the advantage in that a sterilisation apparatus in accordance with the present invention is less likely to short circuit. That is, and as will be appreciated by those skilled in the art, since the plasma generating electrodes of the Sterad apparatus are an integral part of the sterilisation chamber's inner walls, same is more prone to short circuiting due to the metal items to be sterilised coming into contact therewith. This being the reason why the proprietors of the Sterad apparatus state that any metal items must be kept from coming into contact with the sterilisation chamber's inner or internal walls.

The following non-limiting embodiments of a sterilising apparatus in accordance with the present invention will now be described, by way of example, and with reference to, the accompanying drawings in which:

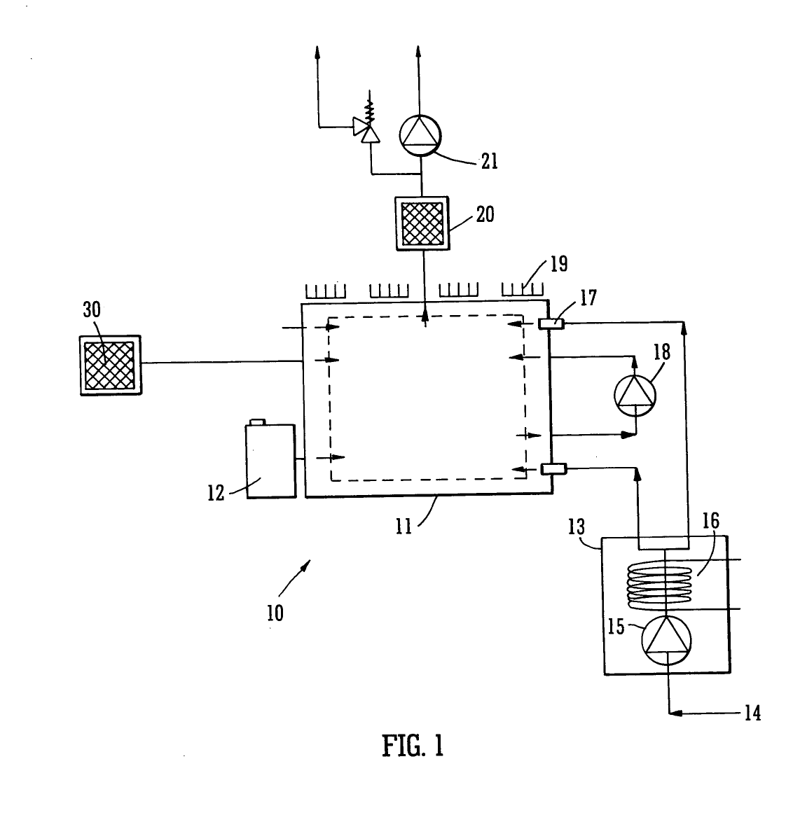

FIG. 1 is a cross-sectional view of a first embodiment of a sterilisation apparatus in accordance with the present invention;

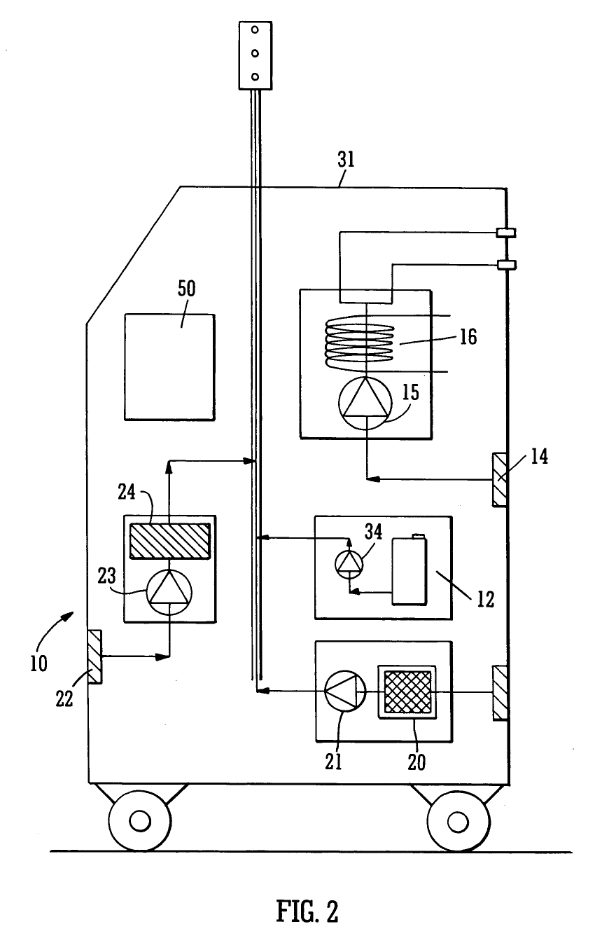

FIG. 2 is a cross-sectional view of a second portable sterilisation apparatus in accordance with the present invention;

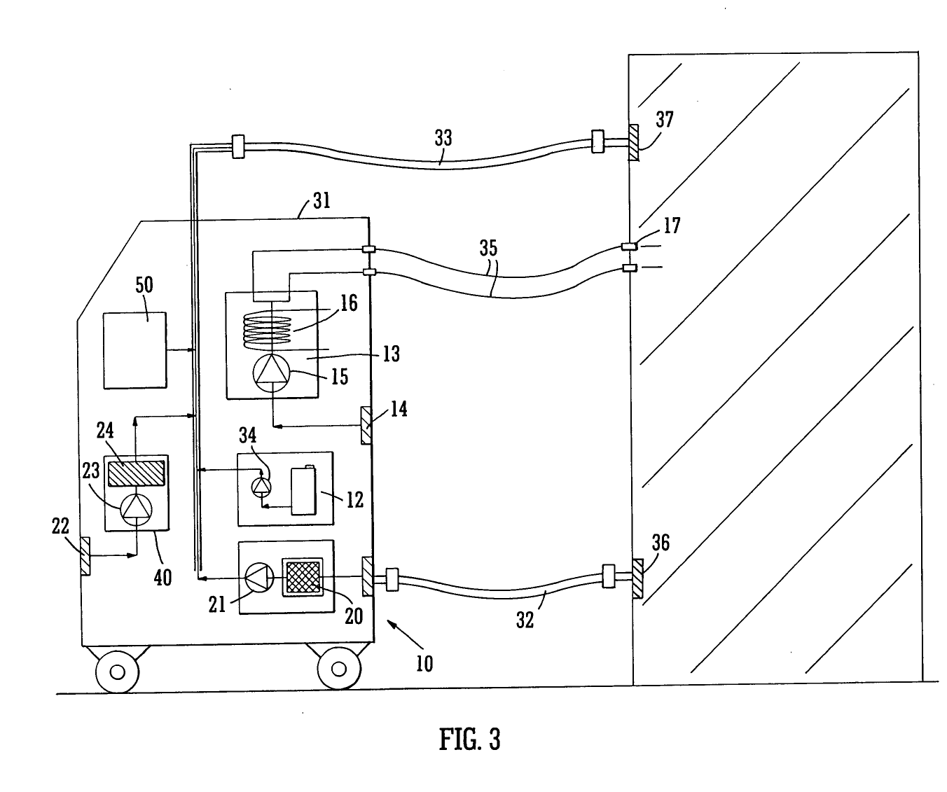

FIG. 3 is a cross-sectional view of the sterilisation apparatus of FIG. 2 when being utilised to sterilise a room; and

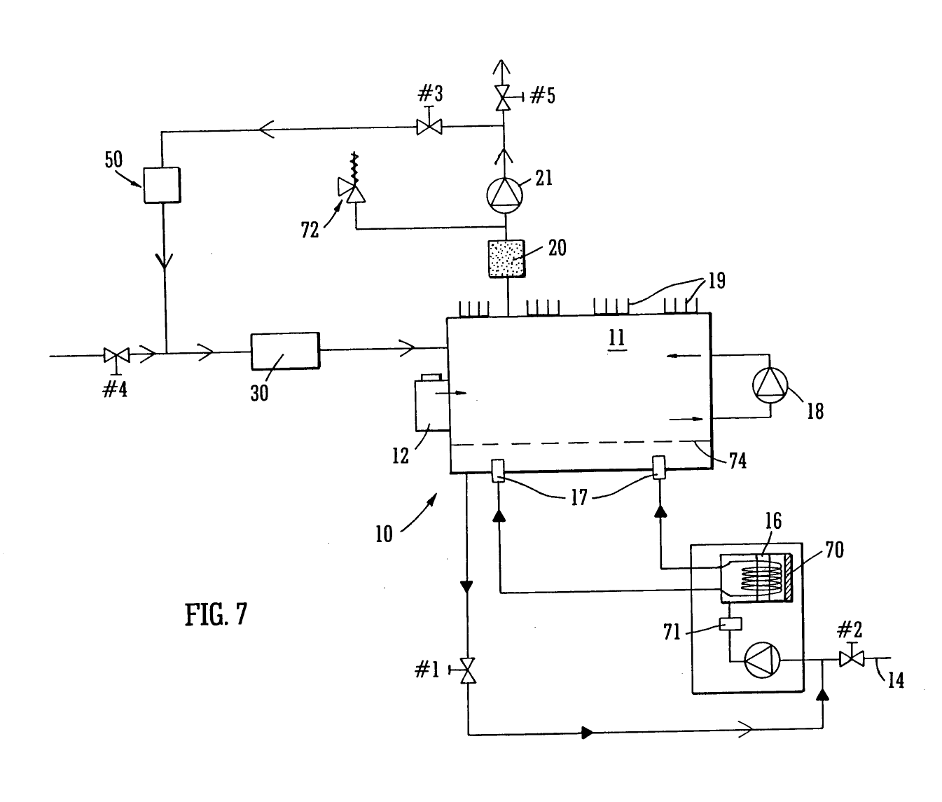

FIG. 7 is a schematic illustration of a third embodiment of a sterilisation apparatus in accordance with the present invention.

As illustrated in FIG. 1, a sterilisation apparatus 10 in accordance with the present invention includes a sterilisation chamber 11 into which the items to be sterilised can be located. During our investigations (see below), the sterilisation chamber 11 had a volume of 60 litres.

A chemical dosing unit 12 is in communication with the sterilisation chamber 11. In use, the chemical dosing unit 12 dispenses the chosen oxidising agent in a gaseous state thereinto. In this connection, the dosing unit 12 includes a heating element for vaporising the oxidising agent such that same can be introduced into the sterilisation chamber 11 as a gas. Preferably, the heating element is automatically inactivated once all the oxidising agent has been vaporised. In addition, it is to be understood that the chemical dosing unit 12 may include an ultrasonic, or equivalent, nebuliser, that is, so the oxidising agent utilised can be introduced in the form of a spray.

Within the sterilisation chamber 11 there are two plasma electrodes 17 that are attached to a plasma generator 13, which includes an air inlet 14, an air pump or compressor 15 for drawing air into the plasma generator 13 and a HF transformer 16. It is to be understood that the number of plasma electrodes 17 may be varied depending on the volume of the sterilisation chamber or area to be sterilised, for example, if the sterilisation chamber or area to be sterilised is large, then there may be 4-6 plasma electrodes. Preferably, the electrodes 17 enter the sterilisation chamber 11 via isolated and sealed apertures. Preferably, the compressor 15 delivers air to the HF transformer 16 at a pressure of 6 bar and flow rate of 12 litres per minute.

The sterilisation chamber 11 is further provided with, or connectable to, a re-circulation system 18 for pumping or re-circulating the air within the sterilisation chamber 11. Once the oxidising agent has been added, it is preferable that the re-circulation system remain activated for the whole of the sterilisation cycle.

The sterilisation chamber 11 is further associated with heat absorption cooling modules 19, which, in use, are used to cool the sterilisation chamber 11 such that the temperature within the chamber 11 can be maintained at a desired level.

The sterilisation chamber 11 is further associated with an extraction system including an activated carbon filter 20 and an air pump 21. In use, and after the generation of plasma, any harmful residue can be neutralised by extraction through the carbon filter 20. In this connection, and so that the sterilisation chamber can be flushed with clean air, same is connected to an ULPA filter 30. It is to be understood that a HEPA filter can be used instead of, or in addition to, the ULPA filter 30.

In order to test the efficacy of the sterilisation process of the present invention against bacterial spores, the sterilisation chamber 11 was loaded in accordance with ISO 11138, BS EN 866, EN ISO 14937 (draft), BS EN 1174, ISO 14161 (draft) with Bacillus stearothermophilus spores prepared on stainless steel carriers having a count of 10⁶.

Formalin gas was then introduced from the chemical dosing unit 12 into the sterilisation chamber 11.

The temperature, which was previously ambient, was increased from 22° C. to 45° C., and the formalin gas was re-circulated via the re-circulation system 18 for 15 minutes within the chamber 11.

The plasma generator 13 was then activated and the gas plasma produced thereby was introduced into the sterilisation chamber such that the gas plasma and formalin could be re-circulated within the sterilisation chamber for the designated time periods tabulated in Table 1 below. During plasma generation, the temperature within the sterilisation chamber 11 was increased to 50° C. and maintained to within +/−3° C. thereof by the heat absorption cooling modules 19.

Once the plasma generator 13 was inactivated, the air pump 21 of the extraction system was activated thereby drawing clean air into the sterilisation chamber 11 via the ULPA filter 30 and discharging the neutralised residue back into the lab via an activated carbon filter 20. Such extraction and neutralisation lasted for 5 minutes.

The sterilisation chamber's door was then opened and each carrier was removed individually with sterile forceps and placed immediately into a separate bottle of Tryptic Soy Broth (TSB). As will be appreciated, TSB is an effective recovery medium, which includes amino acids such as tryptophan for neutralising any residual formaldehyde within a partially surviving cell.

The bottles were then incubated in a water bath for 5 days at 56° C. and readings were taken from them and tabulated as shown in Table 1 below. In this connection, if the contents of a bottle was clear then the sterilisation process was taken to be a success and if the contents of a bottle were turbid, then the sterilisation process was taken to have failed. As part of the control, unexposed steel carriers were included with each batch to be tested.

In addition, the spore carriers were tested either unwrapped (‘nude’) and hence, directly exposed to the formalin vapour-gas plasma sterilisation process, or wrapped and sealed. In this connection, wrapping material included either special Tyvek® sterilising bags (base material manufactured by DuPont Products S.A.) with Tyvek® sterilisation envelopes or paper sterilisation envelopes.

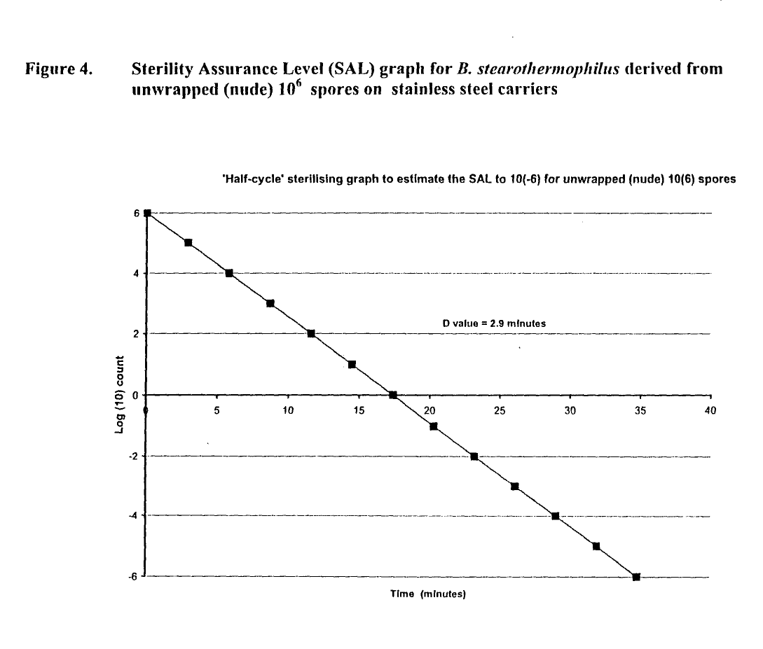

Results gained with the 10⁶ spores are given in Table 1 and FIGS. 4-6.

The method utilised by us was validated by placing 9 spore carriers in the chamber 11 without any gas plasma being produced. It was found that all 9 spore carriers grew at day 1 in TSB (Table 1). Thereafter, one spore carrier that had not been exposed to gas sterilisation was included with each batch of spore carriers placed in gas sterilisation and it was observed that the unexposed spore carriers were always found to yield growth within 24 hours incubation at 56° C. in the water bath.

As regards the manner in which the ‘D’ values (rate of kill for each logarithms count of spores) were calculated, we utilised the formulas for fraction negative methods (International Standard (ISO) 11138-1, 1994; British Standard BS EN 1174-1:1996; British Standard BS EN 866-1, 1997 and Draft BS EN ISO 14161 Document 97/125276). In addition, the Holcomb-Spearman-Karber procedure was used for unwrapped ‘nude’ spore carriers (Table 1) when non-constant numbers of samples were tested and the limited Spearman-Karber procedure was used for wrapped 10⁶ spores (Table 1) when testing was carried out in batches of 8.

The D value (the time to kill 90% of organisms) for 10⁶ Bacillus stearothermophilus spores on “nude” stainless steel carriers was 2.9 minutes, and was 4.3 minutes for those wrapped and sealed in Tyvek® envelopes.

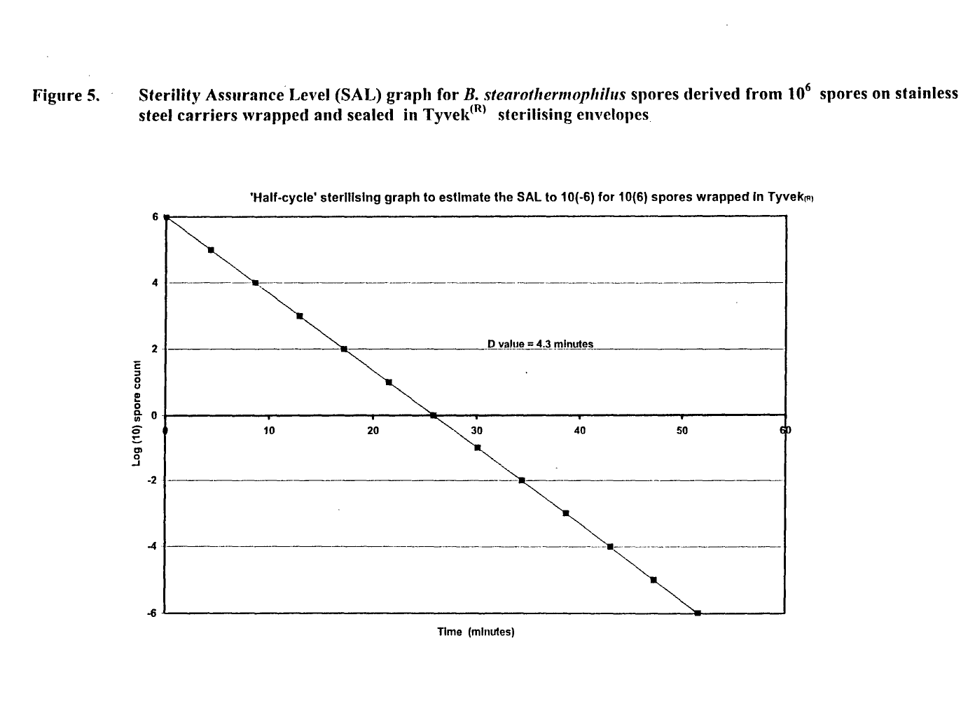

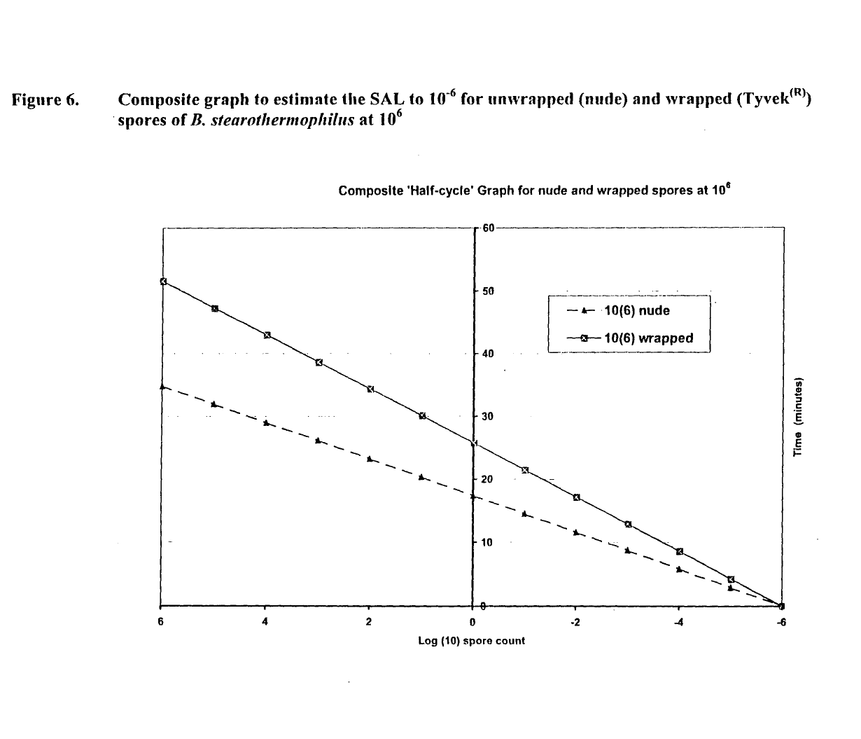

As will be appreciated, a sterilising process should achieve at least a 10⁻⁶ Sterility Assurance Level (SAL) to be sure that the chance of a surviving organism is less than 1 in 10⁶ replicates exposed in the sterilisation chamber at the same time. In practice, this is achieved by constructing a graphical presentation of the Half-Cycle method of steriliser validation for ‘nude’ spores (see FIG. 4) and for wrapped spores (see FIG. 5) with a composite graph (see FIG. 6). The half-cycle time is illustrated for an exposure of 10⁶ spores based on the D values calculated above. The full cycle time is then taken as twice the half-cycle time giving a SAL of 10⁻⁶, that is, of course, assuming that a spore count not greater than 10⁶ is placed in the sterilisation load in the sterilisation chamber 11. If a higher count of spores than this figure is present, then the sterilising cycle time needs to be extended accordingly. If the microbial and spore load is less than 10⁶ then the sterilising time can be reduced. It is thus useful to know the approximate bacterial count expected on items to be sterilised in order to calculate an appropriate time to achieve a SAL of 10⁻⁶.

As will be appreciated, our results show that the gas plasma process has penetrated very effectively through the Tyvek® plastic wrapping film to sterilise the spores contained within it. In addition, our investigations have shown that the gas plasma process of the present invention penetrates effectively through paper envelopes manufactured for medical sterilisation.

In addition, reliability has been demonstrated by repeated experiments demonstrating zero growth out of 8 replicates (Fraction Test Method) after 25, 30, 35 and 40 minutes of testing with gas plasma for nude (unwrapped) spore carriers at 10⁶ B. stearothermophilus and after 35, 40, 45 and 60 minutes for wrapped spore carriers (Table 1). Reliability has also been demonstrated with the vegetable bacteria. (see below)

TABLE 1
RESULTS OF BACTERIAL SPORE TESTS with B.
stearothermophilus spores on stainless steel carriers

| | | GROWTH BY DAY 5 | |
|---|---|---|---|
| | | IN Tryptic Soy Broth | |
| | | | Spore carriers |
| | Total | | wrapped and |
| | Exposure | Nude | sealed in |
| | time | spore | Tyvek ® |
| Tests with 10⁶ spores | (minutes) | carriers | envelopes |
| Unexposed to Formalin Gas | 0 | 9/9 | 9/9 |
| Plasma Sterilisation | | | |
| Vaporised Formalin for 15 mins. + | 30 | 8/8 | — |
| Gas Plasma for 0 mins. + | | | |
| purging for 15 mins. | | | |
| Vaporised Formalin for 15 mins. + | 35 | 8/8 | |
| Gas Plasma for 5 mins. + | | | |
| purging for 15 mins. | | | |
| Vaporised Formalin for 15 mins. + | 40 | 8/8 | |
| Gas Plasma for 10 mins. + | | | |
| purging for 15 mins. | | | |
| Vaporised Formalin for 15 mins. + | 45 | 8/8 | — |
| Gas Plasma for 15 mins. + | | | |
| purging for 15 mins. | | | |
| Vapourised Formalin for 15 | 48 | 1/8 | |
| mins. + Gas Plasma for 18 mins. + | | | |
| purging for 15 mins. | | | |
| Vapourised Formalin for 15 | 50 | 3/8 | |
| mins. + Gas Plasma for 20 mins. + | | | |
| purging for 15 mins | | | |
| Vaporised Formalin for 15 mins. + | 55 | 0/8 | 5/8 |
| Gas Plasma for 25 mins. + | | | |
| purging for 15 mins. | | | |
| Vaporised Formalin for 15 mins. + | 60 | 0/17 | 3/8 |
| Gas Plasma for 30 mins. + | | | |
| purging for 15 mins. | | | |
| Vaporised Formalin for 15 mins. + | 65 | 0/8 | 0/8 |
| Gas Plasma for 35 mins. + | | | |
| purging for 15 mins. | | | |
| Vaporised Formalin for 15 mins. + | 70 | 0/8 | 0/8 |
| Gas Plasma for 40 mins. + | | | |
| purging for 15 mins. | | | |
| Vaporised Formalin for 15 mins. + | 75 | — | 0/16 |
| Gas Plasma for 45 mins. + | | | |
| purging for 15 mins. | | | |
| Vaporised Formalin for 15 mins. + | 90 | — | 0/8* |
| Gas Plasma for 60 mins. + | | | |
| purging for 15 mins. | | | |

*Standard paper envelopes used for sterilisation wrapping instead of Tyvek ®.

In order to meet the requirements of BS EN 556, a typical hospital load was selected. In this connection, and for the purpose of establishing process effectiveness, one load contained a high level of metals (“all metal load”) and another load was made up of mixed materials, namely, metals, plastics and composites (“mixed load”).

Eight spore carriers, each containing 10⁶ spores, were mixed within each load of materials for testing in the gas plasma steriliser 10.

The “all metal load” contained hip prosthesis, spinal screws and plates, as well as arthroscopy shaving instruments and scalpels. The load was challenged with 8×10⁶ Bacillus stearothermophilus stainless steel carriers and sealed within a Tyvek® bag.

A similar metal content was used for the “mixed load” adding a dialysis blood line kit, which contained a range of plastic materials as well as miscellaneous tubing found in hospital suction and infusion equipment. The mixed load was once again sealed inside a Tyvek® bag and challenged as per the “all metal load”. Each load was treated by exposure to 15 minutes of formalin, 60 minutes of gas plasma/formalin mixture and 15 minutes of purging.

Each experiment showed no growth of 8/8 carriers after 5-day incubation period in TSB media, whereas all controls grew on day one.

With 24/24 ‘no growth’ results for each of the two types of load applied, it is evident that the process of the present invention is not susceptible to sterilisation failure due to metal shielding or other undesirable effects; even if any metal objects placed inside the chamber 11 are in direct contact with the chamber's inner walls.

In light of our investigations, we have found the following materials are all satisfactory for use with the process of the present invention.

Metals:

Stainless Steel (300 series), Brass, Aluminium (6000 series), Titanium

Plastics & Rubber:

Nylon, Latex Rubber, Polycarbonate, Polyethylene, Polypropylene, Teflon, PVC, Neoprene, Silicone, Delrin (black), Acrylics and Polystyrene

Other:

Glass, Glass Fibres, Ceramics, miscellaneous bonded materials using cyanoacrelates and UV-cure epoxies as the bonding agents.

In order to test the efficacy of the present process against vegetative bacteria, six types of vegetative bacteria (Staphylococcus aureus, MRSA, coagulase-negative staphylococci, E. coli, Ps. aeruginosa and Salmonella typhimurium) and the yeast, Candida albicans, were tested by preparing cultures at 10⁶/ml and then producing dried preparations on glass slides with at least 10⁴ cfu's (colony forming units).

The slides were immediately exposed in batches of 8, with unexposed controls, to the process within the sterilisation chamber 11 for a total of 45 minutes (15 minutes formalin vaporisation, 15 minutes formalin/gas plasma sterilisation, 15 minutes purging).

Immediately after sterilisation, the slides with bacteria were placed upside down on blood agar plates, and the slides with yeast were placed upside down on Sabouraud agar plates. The bacteria and yeasts grew as colonies on the agar under the glass slide. Plates were incubated for 24 hours at 37° C. in air. Unexposed control cultures grew 10⁴ cfu's.

With reference to the tabulated results (see Table 2 below) all six types of vegetative bacteria and C. albicans, each replicated 8 times, were killed by the sterilisation process of the present invention.

In addition, 56 out of 56 of the slides tested simultaneously in the gas plasma chamber 11 were killed thereby demonstrating the reliability and effectiveness of this process.

Table 2 Results of Testing Vegetative Bacteria and Fungi

Test results for unwrapped ‘nude’ 10⁴ organisms on glass slides exposed to formalin vapour 15 minutes, formalin-gas plasma sterilisation for 15 minutes and purging for 15 minutes (total exposure time of 45 minutes)

| | Failure to |
|---|---|
| Pure cultures at 10⁴ cfu | grow |
| Methicillin-resistant Staphylococcus aureus (MRSA) | 8/8 |
| Oxford Staphylococcus aureus | 8/8 |
| Coagulase-negative staphylococcus (CNS) | 8/8 |
| Escherichia coli | 8/8 |
| Pseudomonas aeruginosa | 8/8 |
| Salmonella typhimurium | 8/8 |
| Candida albicans | 8/8 |
| 56 slides tested simultaneously in the sterilisation chamber | 56/56 |

Secondly, each type of culture was mixed with 10⁶/ml Candida albicans to simulate a heavy organic load to investigate if the presence of the organic matter would seriously interfere with the formalin vapour-gas plasma sterilisation method as exhibited with phenolic disinfection of typhoid bacilli (Chick-Martin modification [1934] of the Rideal-Walker Test [1904]). Dried preparations of the mixtures of organisms, with a minimum of 10⁴ cfu's of each, were made on glass slides immediately before testing. Slides were exposed in the same manner as outlined above (15 minutes formalin, 15 minutes purging) and then processed in the same manner as on the above.

As tabulated in Table 3 below, a minimal effect only, with survival of one to three colonies (cfu's) in two cultures out of 7 from a count of 10⁴, was demonstrated with 15 minutes of formalin vapour-gas plasma. In this connection, and from the bacterial spore experiments above, it would be expected that this level of survival would be totally ablated by extending the period of gas plasma sterilisation from 15 to 30 minutes. These results show that the presence of a high load of organic matter had no detrimental effect on the sterilisation process of the present invention.

Table 3 Results of Testing Vegetative Bacteria and Fungi with an Organic Load

Test results for unwrapped ‘nude’ 10⁴ organisms mixed with 10⁴ Candida albicans on glass slides exposed to formalin vapour 15 minutes, formalin-gas plasma sterilisation for 15 minutes and purging for 15 minutes (total exposure time of 45 minutes)

| Each culture at 10⁴ mixed with C. albicans at 10⁴ | Failure to grow |
|---|---|
| Methicillin-resistant Staphylococcus aureus (MRSA) | 8/8 |
| Oxford Staphylococcus aureus | 8/8 |
| Coagulase-negative staphyloccus (CNS) | 8/8 |
| Escherichia coli | 8/8 |
| Pseudomonas aeruginosa | 8/8 |
| Salmonella typhimurium | 8/8 |
| Mixture af all 6 bacteria above, each with 10⁴ cfu, and | 8/8 |
| C. albicans at 10⁴ cfu | |

In addition to the above, batches of 8 seeded glass slides with 10⁴ cfu's for each of the 7 pure cultures of organisms were wrapped and sealed in Tyvek® envelopes and the experiment repeated in the manner described above.

As shown in Table 4, the gas plasma process fully penetrated through the sealed Tyvek® envelopes. In addition, there was total kill in two batches each of 28 slide tests conducted simultaneously, again demonstrating the reliability and effectiveness of this process.

Table 4 Results of Testing Vegetative Bacteria and Fungi

Test results for 10⁴ organisms on glass slides wrapped and sealed in Tyvek^{(R)} sterilising envelopes exposed to formalin vapour 15 minutes, formalin-gas plasma sterilisation for 60 minutes and purging for 15 minutes (total exposure time of 90 minutes)

| Pure cultures at 10⁴ cfu sealed in Tyvek ® envelopes | Failure to grow |
|---|---|
| Methicillin-resistant Staphylococcus aureus (MRSA) | 8/8 |
| Oxford Staphylococcus aureus | 8/8 |
| Coagulase-negative staphylococcus (CNS) | 8/8 |
| Escherichia coli | 8/8 |
| Pseudomonas aeruginosa | 8/8 |
| Salmonella typhimurium | 8/8 |
| Candida albicans | 8/8 |
| 28 slides tested simultaneously in the sterilisation | 28/28 |
| chamber in two batches | 28/28 |

Testing for the affect of formalin-gas plasma sterilisation on mycobacteria was conducted with M. chelonae. Glass slides were prepared with 10³ cfu's of M. chelonae and were exposed to sterilisation with formalin vapour for 15 minutes, formalin-gas plasma for 30 minutes and purging for 15 minutes. Four controls were not exposed to sterilisation. All slides were inverted on to blood agar and incubated at 37° C for 7 days wrapped in plastic bags. The slides were then removed from each agar plate and the plates re-incubated for a further 7 days. This experiment was repeated for a further 8 slides containing 10³ cfu's of M. chelonae but which were wrapped and sealed within Tyvek® sterilising envelopes.

Results were gained of ‘no growth’ for 8 out of the 8 slides seeded with M. chelonae both exposed ‘nude’ to formalin-gas plasma sterilisation, as well as wrapped and sealed within Tyvek® sterilising envelopes. Control slide cultures grew satisfactorily.

As regards the efficacy of the present process against viruses, we tested same against polio virus (vaccine strain type 2) and herpes simplex virus (type 2); each representative of the two major classes of viruses, namely, hydrophilic and lipophilic respectively. As will be appreciated, it was anticipated that the hydrophilic group would exhibit greater resistance to chemical sterilisation.

The polio virus was prepared in Reece monkey kidney cell lines with an approximate count of 10³ infectious units. The herpes virus was prepared in human embryonic lung cell lines with an approximate count of 10³ infectious units. Cell lines were incubated at 37° C. Viruses were freshly prepared for testing on dry glass coverslips, which were sealed in ampoules. There was a maximum delay of 8 hours between virus preparation and testing. Twelve replicates of each of the two viruses were produced for testing.

The sterilisation (GPS) process was applied as follows:

Six samples of the polio virus and six samples of the herpes virus ampoules were opened and immediately sealed within the Tyvek® sterilisation envelopes. These envelopes were placed in the sterilisation chamber 11 on two occasions.

The first experiment involved exposing 3 samples of polio virus and 3 of herpes to 15 minutes formalin, 30 minutes formalin-gas plasma and 15 minutes purging.

The second experiment involved exposing 3 samples of polio virus and 3 samples of herpes to 15 minutes formalin, 60 minutes formalin-gas plasma and 15 minutes purging.

Two samples of each virus were used for sterilisation (GPS) without wrapping in envelopes. Their ampoule cap was removed and they were placed directly in the sterilisation chamber 11.

One sample of each of polio virus and herpes virus were exposed to 15 minutes formalin, 60 minutes formalin-gas plasma and 15 minutes purging.

One sample of each of polio virus and herpes virus were exposed to 15 minutes formalin, 60 minutes formalin-gas plasma and 15 minutes purging.

Immediately after sterilisation the virus transport media was added and their tops resealed.

Four samples of polio virus and four samples of herpes virus were randomly selected from those produced for testing and defined as controls. These eight ampoules were opened and 5 drops of virus transport media added to each one. They were immediately resealed. This took place at the time that the other samples were being subjected to sterilisation by gas plasma.

The 24 samples were passaged in fresh tissue culture cell lines as described above. All 8 control samples gave cytopathogenic effects (CPE) in their respective cell lines within 2 days for the polio virus and within 4 days for the herpes virus. All 16 virus samples exposed to the differing periods of formalin vapour-gas plasma failed to give virus growth, recognised as CPE, in the tissue culture cell lines after 6 days incubation. Each cell line negative culture was passaged into a fresh cell line of the same type and further incubated for 6 days after which no sterilised (GPS) virus sample yielded any growth. This, we believe, demonstrates, the effectiveness of the present process in killing these two viruses.

As illustrated in FIGS. 2 and 3, a second embodiment of a sterilisation apparatus 10 in accordance with the present invention, which is portable, includes a wheeled housing 31 housing the working components thereof.

As illustrated such apparatus 10, can be connected to, or associated with, a room 11, which, in effect is the sterilisation chamber. That is, anything within the room 11 can be sterilised. Said room being provided with an inlet 37 and outlet 36 to which the working components of the sterilisation apparatus 10 can be attached.

Such apparatus 10 includes a chemical dosing unit 12, which, in use, can dispense the oxidising or sterilising agent, for example, formalin or peracetic acid, in a gaseous state, via a hose or pipe 33, that is, by the action of pump 34, into the room 11 via inlet 37.

Within the room 11 there are plasma electrodes 17, which are attached to a plasma generator 13, via cables 35. Such plasma generator 13 includes an air inlet 14, an air pump 15 for drawing air into the plasma generator 13 and a HF transformer 16. It is to be understood that the room 11 or area to be sterilised can be provided with the plasma electrodes 17 such that the sterilisation apparatus 10 can be simply connected thereto, or that a wall or walls of the room 11 are provided with an aperture through which the plasma electrodes 17 may protrude so that they are suitably located within the room 11. In any event, it is preferable that the plasma electrodes 17 are isolated and sealed such that they cannot come into direct contact with the items to be sterilised.

Air within the room 11 is re-circulated by air pump 21. That is, air is pumped into the room 11 via flexible pipe 33 and inlet 37 and is removed from the room via outlet 36 leading to flexible pipe 32.

The sterilisation apparatus 10, in this embodiment, is further provided with a gas analysing and monitoring support system 50, which, in use, can monitor the process parameters.

As illustrated, the housing may further house an ozone generator 40. Such generator 40 includes an air inlet 22, an air pump 23 and an ozone unit 24. In use, the air pump 23 draws dry air, preferably pure oxygen from a gas canister, into the generator 40 via the air inlet 22 and pumps the air through an air dryer and then through the ozone unit 24 and into the room 11 via flexible pipe 33 and inlet 37. As outlined above, any formalin residue will react with ozone to produce carbon dioxide and water.

The sterilisation apparatus 10 is further provided with an extraction system including an activated carbon filter 20 and an air pump 21. In use, and after the generation of plasma, any harmful residue can be neutralised by extracting the gaseous contents of the room 11, via outlet 36 and flexible pipe 32, through the carbon filter 20.

A non-limiting example of the sterilising process in accordance with the present invention utilising the sterilising apparatus of FIGS. 2 and 3 will now be exemplified below:

A single occupancy hospital ward 11 was artificially seeded with MRSA (Methicyllin Resistant Staphylococcus Aureus) with a 100,000 colony forming units (cfu).

The sterilising apparatus 10 was connected or linked to the ward 11. In this connection, the flexible pipes 33 and 32 were connected to an inlet 37 and outlet 36 provided in the wall of such ward 11 respectively.

The connector cables 35, which are connected to the gas plasma electrodes 17, located within the ward 11, were then connected to the plasma generator 13 of the sterilising apparatus 10. Due to the size of the ward 11 the gas plasma generator 13 was nearly three times the power of the first embodiment outlined above, for example, 600 watts.

Formalin gas was then introduced from the chemical dosing unit 12 into the ward 11, that is, via pipe 33 and inlet 37.

The temperature, which was previously ambient, was increased from 22° C. to 27° C., and the formalin gas was re-circulated via the re-circulation system 18 for 10 minutes within the ward 11.

The plasma generator 13 was then activated and left to run for 3 hours.

Neutralisation or purging was effected by 30 minutes of ozone generation, which was fed into the ward 11 via flexible hose 32 connected to inlet 37, as well as replenishing the air 20 times via the activated carbon filters 20 prior to re-entering the ward 11.

On entering the room, we collected all the randomly sited Staphylococcus and then incubated same for 24 hours. On doing so, no growth was recorded in any of the samples, except the control.

In addition to the above, we have also discovered that the inclusion of a third stage, as described below, can also sterilise items that may be infected with the causitive agent at Creutzfeldt-Jakob Disease (CJD).

Creutzfeldt-Jakob Disease (CJD), the human form of “mad cow” disease, and its variants (vCJD) is a devastating disease which infects the brains of humans.

Investigations have revealed that the infectious agent causing CJD are prions, in particular, TSE prions.

As will be appreciated, there is a real risk of CJD and vCJD being transmitted from patient to patient via re-usable surgical instruments, particularly those used for carrying out tonsillectomy procedures.

This risk clearly identified by the UK national health authority, namely, the NHS, who have issued a mandate to the effect that disposable instruments should be used for forms of surgery where the presence of the causative agent of CJD is thought to be a possibility. As will be appreciated, not only is the use of disposable instruments expensive, but also, in some instances, by the very nature of their disposability they may not be as suitable for a particular application as the re-usable instrument they are intended to replace.

To date, there is no known sterilisation process that is believed to be able to effectively sterilize surgical apparatus and instruments from the causative agent of CJD and its variants, that is, so that same may be re-used. This being the reason why, at present, the standard protocol involves incinerating any surgical apparatus or instruments that may have come into contact with the infectious agent in question, namely, prions.

It is believed that for the first time, that the present invention provides a sterilisation process that can sterilise articles contaminated with the infectious agent of CJD and vCJD. That is, it is believed that our investigations have established that the process of the present invention achieves the International sterilisation standard stipulated by 10 log 6 SAL (Sterility Assurance Limit) even when the articles being sterilised are contaminated with prions.

The process stages of this embodiment of the present invention, which include Stages 1 and 2 outlined above, will now be described by way of example and with reference to FIG. 7.

### Stage 1

As above, Stage 1 involves the addition of an oxidising agent into the sterilisation chamber 11 of the sterilisation apparatus 10. Such oxidising agent starts the sterilisation process by interacting with the many proteins and nucleic acid groups found in the outer coatings, for example, cell walls and cell membranes, of infectious, biological agents, such as bacterium, fungi and viruses etc. also likely to be present on the surgical apparatus or instruments (the “load”) being sterilised. That is, such an oxidising agent affects the integrity of such outer coatings thereby weakening the biological agents defences to the sterilising action of the present process.

In a preferred embodiment, the oxidising agent utilised is peracetic acid (peroxyacetic acid). Preferably, the peracetic acid is used in concentrations of 3-20% ww, preferably 12% or 15% ww, and is added in a ratio of 1-10 ml, preferably 3 ml, per cubic metre of effective sterilisation chamber volume.

As illustrated in the accompanying schematic diagram identified as FIG. 7, the peracetic acid is “atomised” from its fluid container or capsule by means of an ultrasonic, or equivalent, nebuliser 12. That is, it is preferable that the peracetic acid, an oxidising agent, is introduced into the sterilisation chamber in the form of a spray or vapour.

Generally, it takes approximately 1-60 seconds to add the peracetic acid into the sterilisation chamber 11 to achieve the required concentration, outlined above.

Once the oxidising agent is introduced into the sterilisation chamber 11, the chamber's closed loop re-circulating system 18 is activated, such re-circulating system 18 remaining operational throughout the remainder of the process. By way of explanation, and as outlined above, re-circulation has the advantage of attaining total mixing of the gaseous contents within the chamber 11 and/or to assist in pushing any active gas through any long lumen devices present within the load.

Preferably, Stage 1, the pre-treatment stage or “pre-plasma” stage, lasts for approximately 1-20 minutes, preferably 10 minutes, thereby enabling sufficient exposure time of the biological contaminants to the oxidising agent, for example, peracetic acid.

### Stage 2

The second stage of the process of the present invention involves the introduction of a high energy gas plasma field into the chamber 11, preferably via a number of electrodes 17 located at strategic points within the chamber 11, such electrodes preferably being shielded from direct contact with the sterilisation load, that is, under a gas diffusion plate 74.

As outlined above, the gas from which the plasma is derived may be from ambient air, with or without the presence of other gases, and with or without oxygen concentrators.

The ambient air is suitably dried and introduced into the plasma generator 13 via a high pressure pump 15, which is capable of maintaining continuous and stable pressures of between about 4-8 bar, preferably 6 bar, and a continuous flow rate of the order of 5-20 litres per minute, preferably 12 litres per minute.

As illustrated, the flow and pressure monitoring equipment within the plasma loop circuit 70 further modifies the air-flow rate thereby reducing the chamber introduced pressure to minimal positive levels, that is, if the system is operated from external air/gas supply. In addition, the plasma loop circuit 70 includes the required electronic devices and HT-RF transformers 16 capable of applying the required operational voltages of the order of 12,000-18,000 volts, preferably 14,000 volts, at frequencies of the order of 12-100 kHz, preferably 30-40 kHz, such parameters being variable to suit specific process requirements.

As a preferred additional feature, the plasma loop circuit 70 further includes a secondary frequency generating facility, which can be used to extend the plasma energy and penetration range, is also included to operate in the infra red to nanometer range. It is believed that by overlaying a secondary frequency, that is, in addition to the radio frequency described in the KHz range above, it is possible to move the free atoms and other elements of the plasma gas a lot further in terms of distance thereby increasing the range of the plasma generated about an electrode.

The gas plasma introduced or generated within the chamber 100, has plasma characteristics that are fully dependent on external criteria, such as pressure, current, power and the frequency of the created electromagnetic field, electrode system, geometry, as well as the chemical composition of the gases used. The gas is supplied to the electrodes 17 at a temperature slightly greater than ambient, the temperature rise being the function of the pressure generator and the electric arc at the electrodes.

The maximum temperature at the point of plasma discharge preferably does not exceed 80° C. and for temperature sensitive applications, the temperature within the chamber 100 can be controlled by external cooling which maintains the chamber temperature constant in the order of 45-50° C. or as required.

The systems biocidal properties are derived by means of plasma radiation and free radicals resulting by the plasma process breaking down the chemical phase molecules into free radicals etc.

Extensive biocidal testing of this plasma format has established that the effective range of electrons/free radicals created by the process is substantially greater than the visible plasma field which projects few centimetres beyond the electrode discharge point and it would appear to be proportional to the power input and frequency conditions. To this end, in general, a ratio of chamber volume to plasma power equals 0.1 m³ per 100 watts of plasma power. Based on these observations, it will be appreciated that the size of the sterilisation chambers can be theoretically infinite, as long as suitably prepared plasma-generating units, with or without the chemical phase, are multiplied by a similar order of magnitude.

In addition, with the plasma conditions introduced into the sterilisation chamber 11 already primed with the presence of peracetic acid molecules, a complex interaction is formed creating a range of free radicals through plasma interaction with peracetic acid.

Preferably, this second stage of the sterilisation process runs for a period of 5-45 minutes, preferably 20 minutes.

### Stage 3

In most situations, and as outlined above, Stages 1 and 2 would be sufficient to destroy most types of biological contaminants, including spores (see experimental data presented above and below). However, we have discovered that by introducing at least one further sterilisation agent/oxidising agent/biocide directly into the gas stream feeding the plasma electrodes 17, via dosing unit 71, that the causative agents of CJD and vCJD, namely, prions, can also be destroyed. Put another way, it has been discovered by us that by supplementing the gas stream from which the plasma is generated with at least one sterilising agent greatly enhances the effectiveness thereof. To this end, it is believed that the resulting free radicals and stable atoms generated can target prions, which are extremely small.

To this end, it is preferable that a “cocktail” of sterilisation agents or biocidal molecules are added. In this connection, we have discovered that a mixture of hydrogen peroxide (30-50% ww), chlorine/hypochllorate (2-10% ww), iodine and other suitable aldehydes, for example, formalin, are particularly effective. However, it is to be understood that any one of the aforementioned chemicals can be used in combination or alone.

Preferably, the total quantity of the “cocktail” introduced is in the order of 1-15 ml., preferably 3 ml., which is injected directly into the air/gas stream over a period of 1-60 seconds thus quickly mixing to aerosol format with the said air/gas stream and capable of producing high energy free radicals via the plasma electrodes 17 directly into the sterilisation chamber 11.

In some situations, additional (positive) overpressure may be required inside the sterilisation chamber. This is achieved by drawing in ambient air or other gases through the plasma generator into the sealed chamber.

This part of the process has been tested together with Stages 1 and 2 on high inoculums category 2 TSE (scrapies) and by using western blotting techniques it was possible to demonstrate high level of reduction. This, we believe, being indicative of the fact that the process of the present invention can effectively sterilise a load contaminated with prions.

Preferably, Stage 3 of the present process is intended to be operable for 10-30 minutes, preferably 15 minutes, and in extreme situations, can be repeated more than once. To this end, it is preferable that stage 3 begins when it is apparent that the interaction between the oxidising agent of stage 1 and the plasma of stage 2 is minimal, that is, light of the fact that the former's levels are spent.

### Stage 4

Although not essential, in a preferred embodiment, and as outlined above, it is preferable that the present process includes a fourth stage, namely, a neutralisation or purging stage, to neutralise any harmful chemical compounds that may be present within sterilisation chamber at the end of Stages 1, 2 and 3.

At present, the work done by us so far suggests that there are no harmful residues; however, and for the sake of peace of mind, the present process further includes a purging period of 1-15 minutes wherein the air/gas within the chamber 11 is removed by the action of exhaust pump 21 which drains the purged air/gas through absorption filters 20 such as a charcoal filter bed and any air displaced from the chamber 11 is replenished with clean filtered air introduced into the chamber 11 via HEPA or ULPA filters 30. To this end, gas monitoring probe 50 monitors the composition of the purged air.

In addition to the above, additional experimental data illustrating the effectiveness of the process in accordance with the present invention is presented hereinbelow.

### Experimental Data

A) Schedule of Cultures Used.

Multiple cultures were used with 10 log 4 to 10 log 8 loading, with and without additional organic load, and with and without outer wrapping in sealed sterilisation pouches.

B) Vegetative Bacteria and Fungi:

Oxford Staphylococcus/Methicillin resistant Staphylococcus aureus MRSA)/Coagulise negative Staphylococcus (CNC)/Streptococcus faecalis/Vancomycin resistant Enterococcus faecalis (VRE)/Escherichia coli/Candida albicans/Salmonella typhimurium/Pseudomonas aeruginisa.

Clostridial spores—10 log 8

Mycelial Fungi—10 log 6

Viruses: Poliovirus (vaccine strain/type 2) & Herpes Virus—10 log 4

Parasites: Leshmania alderi—10 log 6

B. Stearothermophilus spores—10 log 6

Scrapies—category 2 TSE—10 log 6

Results:

Process stage 1: partial success, good downgrading of vegetative bacteria and viruses, less effective on spores. Minimal effect on sealed cultures inside sterilisation pouches.

Process stage 2: Good on all cultures wrapped and unwrapped other than spores and scrapies.

Process stage 3: Good on all in either format (nude or wrapped).

Process stages 1 & 2: Total duration 30 minutes. Good on all in either format (nude or wrapped).

Process stages 1, 2 & 3—total duration 45 minutes: Absolute sterilisation achieved under the SAL standard definitions for nude and wrapped cultures.

1. A sterilisation process which is not carried out in a vacuum including the steps of:
introducing an oxidising agent in a gaseous or vaporised state into a sterilisation chamber or area to be sterilised; and
introducing gas plasma into the sterilisation chamber or area to be sterilised.

2. A sterilisation process as claimed in claim 1, wherein the oxidising agent is an aldehyde.

3. A sterilisation process as claimed in claim 2, wherein the aldehyde is formalin.

4. A sterilisation process as claimed in claim 3, wherein the formalin has a concentration of 10-40% ww.

5. A sterilisation process as claimed in claim 4, wherein the formalin has a concentration of 35% ww.

6. A sterilisation process as claimed in claim 3, 4 or 5, wherein 1-10 ml of formalin is introduced per cubic metre of effective sterilisation chamber volume or area to be sterilised.

7. A sterilisation process as claimed in claim 6, wherein 3 ml of formalin is introduced per cubic metre of effective sterilisation chamber volume or area to be sterilised.

8. A sterilisation process as claimed in claim 1, wherein the oxidising agent is peracetic acid.

9. A sterilisation process as claimed in claim 8, wherein the peracetic acid has a concentration of 3-20% ww.

10. A sterilisation process as claimed in claim 9, wherein the peracetic acid used has a concentration of 12% ww.

11. A sterilisation process as claimed in any one of claims 7 to 10, wherein 1-10 ml of peracetic acid is introduced per cubic metre of effective sterilisation chamber volume or area to be sterilised.

12. A sterilisation process as claimed in claim 11, wherein 3 ml of peracetic acid is introduced per cubic metre of effective sterilisation chamber volume or area to be sterilised.

13. A sterilisation process as claimed in any one of claims 2 to 7, wherein the sterilisation process, subsequent to the introduction of plasma, further includes the step of introducing ozone into the sterilising chamber or area to be sterilised.

14. A sterilisation process as claimed in any one of the preceding claims, further including the step of neutralising any harmful residue by passing same through a carbon filter.

15. A sterilisation process as claimed in any one of the preceding claims, wherein prior to introducing plasma into the sterilisation chamber or area to be sterilised, the oxidising agent is re-circulated within the sterilising chamber or area to be sterilised for a period of 5 to 15 minutes.

16. A sterilisation process as claimed in claim 15, wherein the oxidising agent is re-circulated for a period of 10 minutes.

17. A sterilisation process as claimed in any one of the preceding claims, wherein gas plasma is introduced into the sterilisation chamber or area to be sterilised for 5-180 minutes.

18. A sterilisation process as claimed in claim 17, wherein the plasma is introduced for 20 minutes.

19. A sterilisation process as claimed in any one of the preceding claims, wherein during the duration of the plasma introducing stage, the gas within the sterilisation chamber or area to be sterilised is re-circulated.

20. A sterilisation process as claimed in any one of the preceding claims, wherein during the introduction of plasma, the pressure within the sterilisation chamber or area to be sterilised is maintained between 1-100, preferably 10 pascals.

21. A sterilisation process as claimed in any one of the preceding claims, wherein during the introduction of plasma the temperature within the sterilisation chamber or area to be sterilised is maintained from 25° C. to 66° C.

22. A sterilisation process as claimed in 21, wherein during the introduction of plasma the temperature within the sterilisation chamber or area to be sterilised is maintained at 50° C. or within +/−3° C. thereof.

23. A sterilisation process as claimed in any one of the preceding claims, wherein the temperature within the sterilisation chamber or area to be sterilised prior to the introduction or production of plasma is maintained from 22° C. to 45° C.

24. A sterilisation process as claimed in any one of the preceding claims, further including the step of adding at least one sterilising agent into the gas stream from which the gas plasma is to be generated.

25. A sterilisation process of claim 24, wherein the at least one sterilising agent is added after 20 minutes of plasma production.

26. A sterilisation process as claimed in claims 24 or 25, wherein the at least one sterilising agent is selected from the group consisting of hydrogen peroxide, iodine, chlorine hypochlorates and aldehydes.

27. A sterilisation apparatus when used to carry out the sterilisation process as claimed by any one of the preceding claims, the sterilisation apparatus being provided with a sterilisation chamber or being connectable to an area to be sterilised and including:
means for providing the sterilisation chamber or area to be sterilised with an oxidising agent in a gaseous or vapour state; and
means for introducing or generating plasma within the sterilisation chamber or area to be sterilised.

28. A sterilisation apparatus as claimed in claim 27, wherein the sterilisation apparatus further includes means for re-circulating the air within the sterilisation chamber or area to be sterilised.

29. A sterilisation apparatus as claimed in any one of claims 27 or 28, further including means for neutralising any harmful residue within the sterilisation chamber or area to be sterilised.

30. A sterilisation apparatus as claimed in claim 29, wherein the means for neutralising the harmful residue, subsequent to the production of plasma, include ozone producing means which dispense ozone into the sterilisation chamber or area to be sterilised.

31. A sterilisation apparatus as claimed in claim 29 or 30, wherein the neutralisation means include a carbon filter through which air from the sterilisation chamber or area to be sterilised is drawn.

32. A sterilisation apparatus as claimed in claim 29 to 31, wherein the neutralisation means includes means for flushing the sterilisation chamber or area to be sterilised with clean air.

33. A sterilisation apparatus as claimed in claim 32, wherein the means for flushing the sterilisation chamber or area to be sterilised with clean air include a ULPA filter through which air from outside the sterilisation chamber or area to be sterilised is drawn.

34. A sterilisation apparatus as claimed in any one of claims 27 to 33, wherein the sterilisation apparatus is portable.

35. A sterilisation apparatus as claimed in any one of claims 27 to 34, wherein the plasma generating means are isolated and/or sealed such that they cannot come into direct contact with the items to be sterilised.

36. A sterilisation apparatus as claimed in any one of claims 27 to 35, wherein the apparatus further includes means for adding at least one sterilising agent into the means for introducing or generating plasma within the sterilisation chamber or area to be sterilised.

37. A sterilisation apparatus as claimed in claim 28, wherein the means for re-circulating the air within the sterilisation chamber or area to be sterilised are connectable to the opening of a lumen of an article being sterilised such that air/gas can be drawn therethrough.
